(19) United States
(12) Patent Application Publication
(10) Pub. No.: US 2013/0337054 A1
(43) Pub. Date: Dec. 19, 2013

(54) TREATMENT OF EXCESSIVE MENSTRUAL BLOOD LOSS BY INTRAVAGINAL ADMINISTRATION OF LOW DOSES OF ANTIFIBRINOLYTIC OR HEMOSTATIC AGENT

(71) Applicant: ARSTAT Inc., Flemington, NJ (US)

(72) Inventor: Arkady RUBIN, Flemington, NJ (US)

(73) Assignee: ARSTAT Inc., Flemington, NJ (US)

(21) Appl. No.: 13/973,394

(22) Filed: Aug. 22, 2013

Related U.S. Application Data
(63) Continuation of application No. PCT/US2012/022565, filed on Jan. 25, 2012.
(60) Provisional application No. 61/449,377, filed on Mar. 4, 2011.

(51) Int. Cl. A61K 31/195; A61K 45/06

(52) U.S. Cl. CPC A61K 31/195; A61K 45/06
USPC 424/451; 514/561

(57) ABSTRACT

A method and drug delivery device for reducing menstrual blood loss in a female are provided. The method comprises administering intravaginally to the female a therapeutically effective amount of an active agent on a delivery device directly to uterine cavity during a menstrual period, wherein the active agent is any one of an antifibrinolytic agent and a hemostatic agent.

### CROSS REFERENCE TO RELATED APPLICATIONS

This application is a continuation of international application PCT/US2012/022565 filed Jan. 25, 2012, which claims the benefit of U.S. provisional patent application No. 61/449,377 filed Mar. 4, 2011, the contents of which are incorporated by reference.

### TECHNICAL FIELD

The present invention relates to the treatment of female menstrual disorders. More specifically, the present invention relates to the pharmacological treatment of excessive menstrual blood loss. More specifically, the present invention relates to the pharmacological treatment of excessive menstrual blood loss by intravaginal administration of low doses of an antifibrinolytic or hemostatic agent.

### BACKGROUND

Heavy menstrual bleeding affects the lives of millions of women and often requires medical attention and initiation of appropriate therapy.

Heavy menstrual bleeding is defined as menorrhagia when the menstrual blood loss (MBL) exceeds 80 mL per menstrual cycle. One-third of all women report heavy menstrual bleeding at some point in their lives, and in Western countries about 5% of reproductive-aged women seek treatment for it annually.'

Heavy menstrual periods may adversely affect women's daily routines and social activities, work productivity, and overall quality of life. Thus, oftentimes, irrespective of the amount of MBL, a woman with complaints about heavy menstrual periods visits her physician and requests medical assistance. Appropriate therapy, including pharmacological treatments, may then be initiated.

Women with heavy menstrual bleeding are frequently offered off-label use of approved hormonal contraceptives. Due to known safety issues, danazol is rarely considered as a viable pharmacological treatment option. NSAIDs are also used for the treatment of women suffering from excessive menstrual blood loss, particularly when the menstrual periods are painful and/or a woman is clinically diagnosed with dysmenorrhea. Surgical procedures (generally hysterectomy) may be considered for women with severe menorrhagia. Yet, removal of the uterus is a radical treatment option with known undesirable consequences, including loss of fertility, surgical morbidity, as well as entailing high cost. There is limited evidence supporting minimally invasive methods of endometrial destruction as efficacious treatment options for menorrhagia.^{1,2}

Among non-hormonal medications, oral tranexamic acid is considered as a first-line treatment option for the treatment of menorrhagia.⁵ Oral tranexamic acid is marketed in the U.S. as Lysteda® and both within and outside the U.S. as Cyklokapron®. As is reported on the Lysteda label, tranexamic acid is a synthetic lysine amino acid derivative, which diminishes the dissolution of hemostatic fibrin by plasmin. In the presence of tranexamic acid, the lysine receptor binding sites of plasmin for fibrin are occupied, preventing binding to fibrin monomers, thus preserving and stabilizing fibrin's matrix structure.³ The antifibrinolytic activity of tranexamic acid results in inhibition of the dissolution of clots.⁴

For many women, tranexamic acid is an efficacious treatment option that ensures the achievement of MBL reduction targets established by the FDA⁶ in the treatment of menorrhagia. Clinical studies indicated that a 3900 mg/day regimen (marketed in the US as Lysteda) meets those targets and significantly reduces limitations on social, leisure and physical activities.^{3,6}

However, the treatment-induced changes in MBL established in the Lysteda clinical trials may be not satisfactory for some women, and many patients may desire even greater reduction in the amount of menstrual flow. As was noted in the medical review of the Lysteda NDA, less than half (44%) of subjects returned to normal MBL after treatment (i.e., achieved a mean on-treatment MBL of less than 80 mL). There were no statistically significant differences between tranexamic acid and placebo treatment with regard to reduction of large stains, small and large clots as well as for changes in serum ferritin levels.⁶ The latter endpoint is particularly meaningful for women with impaired iron status and/or clinically-diagnosed anemia frequently associated with menorrhagia.

In the evaluation of tranexamic acid in the treatment of menorrhagia performed in 2000 by the European Agency for the Evaluation of Medicinal Products (EMEA), a dose-dependent increase in efficacy was noted. The same review recommended a daily dose of 3-4 g/day and indicated that the risk of gastrointestinal adverse events is increased at 6 g/day.⁷ While the FDA-approved tranexamic acid regimen is within the aforementioned recommended dosing range, certain Warnings and Precautions, e.g., the need for dose adjustment in women with renal impairment; increase in the risk of blood clots, stroke, or myocardial infarction in the event of concomitant therapy with hormonal contraceptives; and the possibility of severe allergic reactions, visual or ocular adverse effects,³ reflect regulatory concerns regarding Lysteda's safety. In the risk-benefit assessment, the FDA medical reviewer suggested a 50% dose reduction for women who do not tolerate the common adverse events associated with the approved treatment regimen.⁶ Taken together, the clinical evidence indicates that the efficacy of oral tranexamic acid in the treatment of menorrhagia must be weighed against the potentially disturbing side effects associated with this medication. When taken via the conventional oral route at approved doses, the drug may raise safety concerns.

In addition to the parenteral drug delivery routes (oral and intravenous), topical administration of tranexamic acid has also been extensively studied. Efficacy of this route was established in a number of clinical studies.^{8,9,10} In various clinical settings, placebo-adjusted differences in blood loss (indicating a direct drug effect) ranged from 55 mL to 750 mL.^{9,11,12,13} The lower limit of the reported range is above the blood loss decrease (50 mL) considered by the FDA as a desirable menstrual blood loss reduction target.⁶

Local administration of tranexamic acid is proven to be efficacious despite very low, sometimes undetectable, circulating drug levels. For example, use of mouthwash containing tranexamic acid was shown to reduce the incidence of postoperative bleeding complications when compared to placebo.⁸ Such an effect was achieved despite a small amount of tranexamic acid being administered and its short residence time in the oral cavity.¹⁴ After administration of an oral tablet of tranexamic acid, mean drug plasma concentrations (7 mcg/mL) reached therapeutic levels, whereas the drug was not detected in saliva samples. After a mouth rinse, plasma concentrations remained below 2 mcg/mL while the saliva concentrations reached a much higher maximum level (above 200 mcg/mL) and remained at a therapeutic level for more than two hours.^{8,15} Reduction of the postoperative blood loss without detectable plasma drug levels was also reported for other locally administered antifibrinolytic drugs, specifically aprotinin.¹¹

A number of studies investigated fibrinolytic enzyme systems in menstrual blood.^{16,17,18,19,20,21,22,23} Results of one of these studies strongly suggest that high levels of menstrual plasminogen activator and plasmin are most likely derived from the endometrium, with significantly greater levels in women with excessive bleeding when compared to those with normal menstrual volumes. Notably, no such differences could be found in the peripheral blood. Both plasminogen activator and plasmin activity in menstrual blood were significantly higher than those in peripheral blood, irrespective of the intensity of the women's menstrual bleeding.¹⁷

Effectiveness of intravaginal drug delivery is supported by a substantial body of evidence. It is established for estrogens used to treat vaginal atrophy and related symptoms²⁴, as well as for osteoporosis and other menopausal symptoms.²⁵ Other examples of compounds efficacious after vaginal administration include (but are not limited to) misoprostol for cervical ripening²⁶, a danazol ring for the treatment of infiltrating endometriosis²⁷, and a progesterone gel.^{28,29} The contraceptive efficacy of levonorgestrel(LNG)-containing intrauterine system, Mirena® with 20 mcg/day LNG delivery is at least comparable to that reported for the LNG-only pill delivering a 50% greater daily dose. As noted in the Mirena NDA Medical review, serum concentrations of levonorgestrel for Mirena are approximately one-tenth the serum concentration produced by an oral contraceptive containing 0.1 mg LNG and about half that produced by the Norplant® system. The local endometrial concentrations, however, are over 100 times higher in Mirena users than in users of oral contraceptives containing 0.25 mg LNG.³⁰

### SUMMARY

Certain embodiments disclosed herein provide a method for effectively decreasing menstrual blood loss without the undesirable side effects of current oral medications, by providing for intravaginal delivery of an antifibrinolytic or hemostatic agent. As disclosed herein, local administration of tranexamic acid (or another antifibrinolytic or hemostatic agent) can be rendered safe and efficacious if it utilizes vaginal drug delivery. Drug delivery devices useful in the method of the present invention allow achieving drug delivery to the affected tissues (e.g., uterine cavity) during menstrual periods. While a vaginal ring is a preferred drug delivery device in the method of the present invention, other delivery devices can also be used, including, without limitation, a vaginal tablet, a pessary, an ovule, a suppository, a vaginal sponge, a diaphragm, a pad, a tampon, foam, cream, ointment, and gel.

According to one embodiment, the active drug (i.e., an antifibrinolytic or hemostatic agent) is delivered directly to the affected tissue(s), particularly the uterine cavity, that are close to the vagina where the delivery device (e.g., vaginal ring) is placed. As specified herein, effective local concentrations of drug are achievable with doses much lower than those administered intravenously, or by the oral route. When used according to the method of the invention, levels of tranexamic acid (or another antifibrinolytic or hemostatic agent) in the systemic circulation are greatly reduced as compared to oral therapies, possibly below detectable limits, leading to a lower incidence of adverse events, such as diarrhea, nausea, vomiting, allergic reactions, disturbance of color, sharpness, or field of vision, etc. The local administration of tranexamic acid may also eliminate risk of systemic toxicity and thrombloembolism, which are well known risks associated with its oral and intravenous administration. Relatively high local tissue concentrations of tranexamic acid (or another suitable antifibrinolytic or hemostatic agent) achievable by the method of the present invention ensure a shorter time to achieving the desired hemostatic changes, including reduction of plasminogen activator and plasmin levels, formation of strong and stable blood clots, etc. The published findings suggest that plasminogen activator and plasmin activity in menstrual blood are significantly higher than those in peripheral blood irrespective of the intensity of the women's menstrual bleeding.¹⁷ Better compliance (thereby avoiding missed pills by women using such a device) is also expected.

Following the local administration of tranexamic acid, reduction of the menstrual blood loss is expected to exceed respective targets established by the FDA⁶ in the treatment of menorrhagia.

For a number of drugs delivered intravaginally, the oral route/intravaginal route ratio for the daily doses seems to be close to 10:1. The same ratio may reasonably be assumed for tranexamic acid (or another antifibrinolytic or hemostatic agent).

While the exact intravaginal doses for each drug useful in the method of the present invention are going to be determined in clinical trials, the possibility of a drastic dose decrease, relative to the currently-approved oral doses, with no compromise (but rather improvement) in the reduction of menstrual blood loss is surprising and new. With an expected decrease in drug-related adverse events, this treatment modality may be considered as the first treatment option in the management of heavy menstrual bleeding. If a woman is not satisfied with the ensuing results, other therapeutic interventions (for example, oral tablets at much greater doses) may be considered. Also surprising and new is the possibility of achieving a therapeutic effect (manifested in a reduction in menstrual blood loss) in the absence of detectable plasma concentration levels, or in the presence of circulating levels of the drug much lower than those reported after administration of oral tablets.

In addition to tranexamic acid, the reduction of menstrual blood loss may utilize a number of antifibrinolytic and hemostatic agents, including ε-amino-caproic acid, aprotinin, antipan, gabexate mesilate, pepstatin, leupeptin, chymostatin and others. The exact dose of these compounds will be determined in future clinical trials. Initial dose selection will be driven by a number of factors, including, but not limited to, the potency of the compound tested, the amount of menstrual flow and the presence of clots, stains in the target population, the severity of the symptoms associated with menstruation, as well as patient characteristics (age, weight, presence of anemia, duration of the disease, etc.).

### DETAILED DESCRIPTION

The embodiments disclosed herein are only examples of the many possible advantageous uses and implementations of the innovative teachings presented herein. In general, statements made in the specification of the present application do not necessarily limit any of the various claimed embodiments. Moreover, some statements may apply to some inventive features but not to others.

### Definitions:

A vaginal ring (also known as an intravaginal ring) is a polymeric drug delivery device providing controlled release of drug(s) to the vagina and uterine cavity over an extended period of time.

Menstrual flow is defined as encompassing menstrual blood and/or menstrual fluid.

A therapeutically effective amount of an antifibrinolytic or hemostatic agent is defined as the amount of a drug that results in a significant (preferably, at least 15%) change in menstrual blood loss when compared to the pre-treatment levels.

Certain disclosed embodiments provide intravaginal delivery of a therapeutically effective amount of an antifibrinolytic or hemostatic agent for the reduction of menstrual blood loss and improvement in related symptoms. This antifibrinolytic or hemostatic agent can be administered to females suffering from excessive menstrual blood loss from the onset of menstrual bleeding until the resolution of related symptoms and/or the end of the menstrual period.

In the method disclosed herein, the antifibrinolytic or hemostatic agent can be delivered using any intravaginal delivery device known in the art. Non-limiting examples of useful delivery devices include a vaginal ring, a vaginal tablet, a pessary, an ovule, a suppository, a vaginal sponge, a diaphragm, a pad, a tampon, foam, cream, ointment, and gel.

In a preferred embodiment, the drug delivery device is a vaginal ring. In one embodiment, the agent can be mixed throughout the vaginal ring. In one embodiment, the agent can be distributed uniformly throughout the vaginal ring. In another embodiment, the agent can be encapsulated in a part of the vaginal ring. In yet another embodiment, the agent can be located at the center of the vaginal ring. In one embodiment, a membrane of the agent can be placed between an un-medicated core and a metering layer of appropriate material. The use of a vaginal drug delivery device delivering the agent directly to the affected tissues is expected to enhance the agent's efficacy in the reduction of the volume of menstrual blood loss and improvement in the related symptoms and a woman's quality of life; it may also result in a shorter duration of menstrual bleeding.

The use of vaginal drug delivery device delivering the agent directly to the affected tissues is also expected to significantly reduce the agent's daily dose when compared to other routes of drug administration; this may result in a lower systemic drug circulation, possibly below detectable levels, and a lower incidence of drug-related adverse events.

In all embodiments, the agent is from a class of drugs called antifibrinolytic agents or hemostatic agents. Non-limiting examples of useful agents include, e.g., tranexamic acid, ε-amino-caproic acid, aprotinin, antipan, gabexate mesilate, pepstatin, leupeptin, chymostatin, and metabolites thereof.

Daily agent doses useful in the drug delivery method disclosed herein do not exceed 1 g. In a preferred embodiment, daily agent doses useful in the drug delivery method disclosed herein range from 50 mcg to 500 mg.

In one preferred embodiment, the agent is tranexamic acid with a daily drug delivery dose ranging from 100 mg to 250 mg. In another preferred embodiment, the agent is ε-amino-caproic acid with a daily drug delivery dose ranging from 350 mg to 500 mg. In yet another preferred embodiment, the agent is aprotinin with a daily drug delivery dose ranging from 250 mg to 400 mg.

In certain embodiments, the disclosed method is used to treat females with menstrual bleeding of less than 80 mL per menstrual cycle. In certain embodiments, the disclosed method is used to treat females with menstrual bleeding of more than 80 mL per menstrual cycle. In certain embodiments, the disclosed method is used to treat females clinically diagnosed with menorrhagia. In certain embodiments, the disclosed method is used to treat females clinically diagnosed with idiopathic menorrhagia.

In certain embodiments, the disclosed method is used to treat females clinically diagnosed with cyclic heavy menstrual bleeding. In certain embodiments, the disclosed method is used to treat females clinically diagnosed with dysfunctional uterine bleeding. In certain embodiments, the disclosed method is used to treat females with no clinical diagnosis related to menorrhagia, idiopathic menorrhagia, cyclic heavy menstrual bleeding, or dysfunctional uterine bleeding, but who perceive their menstrual periods to be heavy. In certain embodiments, the disclosed method is used to treat females clinically diagnosed with anemia.

The present invention is also described and demonstrated by way of the following non-limiting examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The various disclosed embodiments are therefore to be limited only by the terms of the appended claims along with the full scope of equivalents to which those claims are entitled.

### Example 1

The vaginal ring serving as a drug delivery device comprises a supporting ring free of active drug. The next (second) layer contains medication selected for treatment of excessive menstrual blood loss (tranexamic acid or another antifibrinolytic or hemostatic agent). This layer is coated with the third, drug-free layer. Detailed description of such vaginal ring and suitable manufacturing methods can be found in U.S. Pat. No. 4,822,616.

Per U.S. Pat. No. 4,822,616, the supporting ring is made from a physiologically acceptable synthetic resin, such as, e.g., polyethylene, RTV silicone elastomers, LTV silicone elastomers, polyamides and polytetrafluoroethylene. The second layer with active medication comprises a pharmaceutically acceptable resin from which the drug is released. A preferred embodiment consists of the combination of drug and LTV silicone elastomer with a composition also described in the patent. Any LTV silicone elastomer is used in the third layer. The proposed vaginal ring ensures release of the active drug within the limits of the dosage required for the desired reduction of menstrual blood loss.

In one embodiment, the second layer is medicated with tranexamic acid in an amount adequate to release the drug in a rate of 100-250 mg/day. In another embodiment, the second layer is medicated with ε-amino-caproic acid in an amount adequate to release the drug in a rate of 350-500 mg/day. In yet another embodiment, the second layer is medicated with aprotinin acid in an amount adequate to release the drug in a rate of 250-400 mg/day.

### Example 2

The vaginal ring serving as a drug delivery device comprises an active drug selected for treatment of excessive menstrual blood loss (tranexamic acid or another antifibrinolytic or hemostatic agent) and a delivery module. The delivery module comprises (a) a reservoir for storing the active drug, (b) a rate controller or wall that is formed of styrene-butadiene copolymer that maintains the prescribed rate of drug release throughout the life of system, (c) an energy source or the concentration of the active drug in the reservoir that provides the driving means for transferring the active drug from a higher amount in the reservoir to the rate controller, (d) an inner mass transfer conductor for housing the active drug in the reservoir, and (e) a portal that provides the exit from the drug delivery module to the tissues. A detailed description of such vaginal ring and its manufacturing process can be found, for example, in U.S. Pat. No. 4,250,611.

In one embodiment, the delivery module of the vaginal ring contains tranexamic acid in an amount supporting the drug release at a rate of 100-250 mg/day. In another embodiment, the delivery module of the vaginal ring contains ε-amino-caproic acid in an amount supporting the drug release at a rate of 350-500 mg/day. In yet another embodiment, the delivery module of the vaginal ring contains aprotinin in an amount supporting the drug release at a rate of 250-400 mg/day.

### Example 3

The vaginal ring serving as a drug delivery device is a ring-shaped solid carrier made of silicone rubber (polysiloxane) or other suitable material. The ring has a homogenous design with an active drug dispersed in the carrier. Detailed description of such vaginal ring can be found, for example, in U.S. Pat. No. 5,869,081. Per U.S. Pat. No. 5,869,081, the vaginal ring provides sustained release of the medication and results in low circulatory levels of the drug, while concentrating its biological effect on a regional level.

In one embodiment, the carrier contains tranexamic acid in an amount supporting the drug release at a rate of 100-250 mg/day. In another embodiment, the carrier contains ε-amino-caproic acid in an amount supporting the drug release at a rate of 350-500 mg/day. In yet another embodiment, the carrier contains aprotinin in an amount supporting the drug release at a rate of 250-400 mg/day.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

### REFERENCES

¹Heavy menstrual bleeding: Assessing Impact, Evaluating Management Options; Supplement to OBG Management, October 2009; accessed at http://www.obgmanagement.com/PDF/supplOBG heavybleeding.pdf (reference on file)

²Barbara S, Apgar et al. Treatment of Menorrhagia. American Family Physician—Volume 75, Issue 12 (June 2007).

³Lysteda®. Full Prescribing Information.

⁴Joseph Y. Lee, et al. Treatment of Menorrhagia with Tranexamic Acid. J Soc Obstet Gynaecol Can 2000; 22(10):794-8.

⁵National Collaborating Centre for Women's and Children's Health. Heavy menstrual bleeding, London (UK): Royal College of Obstetricians and Gynaecologists 2007 Jan. 7

⁶Center for Drug Evaluation and Research. Lysteda® Medical review; accessed at http://www.accessdata.fda.gov/drugsatfda docs/nda/2009/022430s000medr.pdf (reference on file)

⁷Overall Summary of the Scientific Evaluation of Cycle-f, EMEA, 2000.

⁸Dunn C J, Goa K L. Tranexamic acid: a review of its use in surgery and other indications. Drugs. 1999; 57:1005-32.

⁹Jabalameli M., Zakeri K. Evaluation of Topical Tranexamic Acid on Intraoperative Bleeding in Endoscopic Sinus Surgery Iran J Med Sci December 2006; Vol 31 No 4 221

¹⁰Boethius J. “Method and hemostatic patch for effecting local hemostasis”, U.S. Pat. No. 7,022,125, Issued on Apr. 4, 2006.

¹¹De Bonnis M. et al. Topical use of tranexamic acid in coronary artery bypass operations: a double-blind, prospective, randomized, placebo-controlled study. J Thorac Cardiovasc Surg. 2000 March; 119(3):575-80

¹²Fawzy H, et al. Can local application of Tranexamic acid reduce post-coronary bypass surgery blood loss? A randomized controlled trial. J Cardiothorac Surg. 2009 Jun. 18; 4:25

¹³Vlessides M. Topical Tranexamic Acid Cuts Bleeding After Knee Surgery. Clinical Anesteology. February 2010|VOLUME: 36:2

¹⁴Randall C. U.K. National Health Service. Surgical management of the primary care dental patient on warfarin. March 2007. Accessed at http://www.dundee.ac.uk/tuith/Static/info/warfarin.pdf (reference on file)

¹⁵Sindet-Pedersen S. Distribution of tranexamic acid to plasma and saliva after oral administration and mouth rinsing: a pharmacokinetic study. J Clin Pharmacol 1987; 27:1005-8.

¹⁶Gleeson NC et al. The effect of tranexamic acid on measured menstrual loss and endometrial fibrinolytic enzymes in dysfunctional uterine bleeding. Acta Obstetricia et Gynecologica Scandinavica 1994 73:3, 274-277

¹⁷Dockeray C J. et al. The fibrinolytic enzyme system in normal menstruation and excessive uterine bleeding and the effect of tranexamic acid. Eur J Obstet Gynecol Reprod Biol. 1987 April; 24(4):309-18

¹⁸Rybo G. Plasminogen activators in endometrium. Acta Obstet Gynecol Scand. 1966; 45:411-449

¹⁹Hefnawi A S. et al. Fibrinolytic activity of menstrual blood in normal and menorrhagic women and in women wearing the Lippes Loop and Cu-T (200). Int J Gynaecol Obstet 1979; 16:400-407.

²⁰Hahn L. et al. Blood coagulation, fibrinolysis and plasma protein in women with normal and with excessive menstrual blood loss. Br J Obstet Gynaecol 1976; 83: 974-980.

²¹Cole S K. Clarkson A R. Menstrual blood loss and fibrin degradation products. Br Med J 1972; 1:78-79.

²²Rees M C P et al. Coagulation factors and fibrinolytic proteins in menstrual fluid collected from normal and menorrhagic women. Br J Obstet Gynaecol 1985; 92: 1164-1168.

²³Basu H K. Fibrin degradation products in sera of women with normal menstruation and menorrhagia. Br Med J 1970; 1:74-75.

²⁴Kingsberg S A., et al. Treating dyspareunia caused by vaginal atrophy: a review of treatment options using vaginal estrogen therapy. International Journal of Women's Health. August 2009, Volume 2009:1 Pages 105-111

²⁵Ballagh S A. Vaginal Ring Hormone Delivery Systems in Contraception and Menopause. Clinical obstetrics and gynecology/volume 44/number 1/March 2001

²⁶Iyer V., et al. Vaginal drug delivery. ExpressPharma, 1-15 Jul. 2008

²⁷lgarashi M. Novel vaginal danazol ring therapy for pelvic endometriosis, in particular deeply infiltrating endometriosis. Hum Reprod. 1998 July; 13(7):1952-6.

²⁸Ficicioglu C. et al. High local endometrial effect of vaginal progesterone gel. Gynecological Endocrinology, Volume 18, Issue 5 May 2004, pages 240-243.

²⁹Kuhl H. Pharmacology of estrogens and progestogens: influence of different routes of administration. Climacteric 2005; 8(Suppl 1)3-63.

³⁰Center for Drug Evaluation and Research. Application Number 21-225. Mirena® NDA Medical review; accessed at Mirena® NDA Medical review; accessed at http://www.accessdata.fda.qov/druqsatfda docs/nda/2000/21-225.pdf Mirena Medr.pdf (reference on file)

What is claimed is:

1. A method for reducing menstrual blood loss in a female comprising:
administering intravaginally to the female a therapeutically effective amount of an active agent on a delivery device directly to uterine cavity during a menstrual period, wherein the active agent is any one of an antifibrinolytic agent and a hemostatic agent.

2. The method of claim 1, wherein the delivery device is any one of: a vaginal ring, a vaginal tablet, a pessary, an ovule, a suppository, a vaginal sponge, a diaphragm, a pad, a tampon, foam, cream, ointment, and gel.

3. The method of claim 2, wherein the active agent contained in the vaginal ring is any one of the following forms: mixed throughout the vaginal ring, distributed uniformly throughout the vaginal ring; encapsulated in a part of the vaginal ring, located at the center of the vaginal ring, and placed between an un-medicated core and a metering layer of the vaginal ring.

4. The method of claim 1, wherein each of the antifibrinolytic agent and the hemostatic agent comprises any one of: tranexamic acid, ε-amino-caproic acid, aprotinin, antipan, gabexate mesilate, pepstatin, leupeptin, chymostatin, and metabolites thereof.

5. The method of claim 1, wherein the active agent is delivered at a daily agent dose that does not exceed 1 g.

6. The method of claim 5, wherein the daily agent dose ranges from 50 mcg to 500 mg.

7. The method of claim 6, wherein the active agent is tranexamic acid with the daily dose ranging from 100 mg to 250 mg.

8. The method of claim 6, wherein the active agent is ε-amino-caproic acid with the daily dose ranging from 350 mg to 500 mg.

9. The method of claim 6, wherein the active agent is aprotinin with the daily dose ranging from 250 mg to 400 mg.

10. The method of claim 1, wherein the female has a condition comprising any one of: menorrhagia, idiopathic menorrhagia, cyclic heavy menstrual bleeding, dysfunctional uterine bleeding, and anemia.

11. A method for reducing a volume menstrual blood loss in a female comprising:
administering intravaginally to the female a therapeutically effective amount of an active agent, wherein the active agent is delivered on a delivery device directly to a uterine cavity during menstrual periods to reduce plasminogen activator and plasmin levels in the menstrual blood, thereby reducing the volume of the menstrual blood loss, wherein the active agent is any one of an antifibrinolytic agent and a hemostatic agent.

12. The method of claim 11, wherein the delivery device is any one of: a vaginal ring, a vaginal tablet, a pessary, an ovule, a suppository, a vaginal sponge, a diaphragm, a pad, a tampon, foam, cream, ointment, and gel.

13. The method of claim 11, wherein each of the antifibrinolytic agent and the hemostatic agent comprises any one of: tranexamic acid, ε-amino-caproic acid, aprotinin, antipan, gabexate mesilate, pepstatin, leupeptin, chymostatin, and metabolites thereof.

14. The method of claim 11, wherein the active agent is delivered at a daily agent dose that does not exceed 1 g.

15. The method of claim 11, wherein the active daily agent dose ranges from 50 mcg to 500 mg.

16. The method of claim 15, wherein the active agent is tranexamic acid with the daily dose ranging from 100 mg to 250 mg.

17. The method of claim 15, wherein the active agent is ε-amino-caproic acid with the daily dose ranging from 350 mg to 500 mg.

18. The method of claim 15, wherein the active agent is aprotinin with the daily dose ranging from 250 mg to 400 mg.

19. An intravaginal drug delivery device comprising:
a therapeutically effective amount of an active agent, wherein the delivery device delivers the agent directly to a uterine cavity, the delivery device delivers a daily dose of the active agent which does not exceed 1 g, wherein the active agent is any one of an antifibrinolytic agent and a hemostatic agent.

20. The intravaginal drug delivery device of claim 19, wherein the intravaginal drug delivery device is any one of: a vaginal ring, a vaginal tablet, a pessary, an ovule, a suppository, a vaginal sponge, a diaphragm, a pad, a tampon, foam, cream, ointment, and gel.

21. The intravaginal drug delivery device of claim 19, wherein the active agent is contained in the vaginal ring is any one of the following forms: mixed throughout the vaginal ring, distributed uniformly throughout the vaginal ring; encapsulated in a part of the vaginal ring, located at the center of the vaginal ring, and placed between an un-medicated core and a metering layer of the vaginal ring.

22. The intravaginal drug delivery device of claim 19, wherein each of the antifibrinolytic agent and the hemostatic agent comprises any one of: tranexamic acid, ε-amino-caproic acid, aprotinin, antipan, gabexate mesilate, pepstatin, leupeptin, chymostatin, and metabolites thereof.

23. The intravaginal drug delivery device of claim 19, wherein the active agent is delivered at a daily dose of the active agent that ranges from 50 mcg to 500 mg.

24. The intravaginal drug delivery device of claim 23, wherein the active agent is tranexamic acid with a daily dose ranging from 100 mg to 250 mg.

25. The intravaginal drug delivery device of claim 23, wherein the active agent is ε-amino-caproic acid with the daily dose ranging from 350 mg to 500 mg.

26. The intravaginal drug delivery device of claim 23, wherein the agent is aprotinin with the daily dose ranging from 250 mg to 400 mg.
